(19) United States
(12) Patent Application Publication
(10) Pub. No.: US 2022/0241172 A1
(43) Pub. Date: Aug. 4, 2022

(54) ORAL CARE GUMMIES

(71) Applicant: BASF SE, Ludwigshafen (DE)

(72) Inventors: Denis Ontumi, Tarrytown, NY (US); Natasha Chamberlin, Tarrytown, NY (US); Anna Anita Gripp, Florham Park, NJ (US)

(21) Appl. No.: 17/623,810

(22) PCT Filed: Jun. 22, 2020
(86) PCT No.: PCT/US2020/038928

§ 371 Date: Dec. 29, 2021

Related U.S. Application Data
(60) Provisional application No. 62/871,462, filed on Jul. 8, 2019.

(51) Int. Cl. A61K 8/63; A61K 8/02; A61K 8/65; A61K 8/34; A61K 8/67; A61K 8/73; A61K 8/25; A61K 8/96; A61Q 11/00

(52) U.S. Cl. CPC A61K 8/63; A61K 8/0216; A61K 8/65; A61K 8/345; A61K 8/678; A61K 8/73; A61K 2800/28; A61K 8/25; A61K 8/965; A61Q 11/00; A61K 2800/92; A61K 2800/87; A61K 8/342

(57) ABSTRACT

Disclosed herein is an oral care composition, such as a mouthwash, in a form of an elastic solid, such as a gummy. The mouthwash gummy may comprise a therapeutically effective amount of an active agent for treatment of an oral condition and an excipient. Also disclosed herein are methods of preparing and methods of using said oral care composition to, for instance, cleanse a mouth, treat an oral condition, and/or refresh breath.

### RELATED APPLICATIONS

This application claims priority to U.S. Provisional Patent Application No. 62/871,462, filed Jul. 8, 2019, which is herein incorporated by reference in its entirety.

### FIELD OF THE INVENTION

The present invention relates to the field of oral care compositions, in particular, to mouthwash composition in the form of a gummy, methods of preparation thereof, and methods of use thereof.

### BACKGROUND OF THE INVENTION

Traditional oral care formulations mostly consist of dentifrice and mouthwash. The dentifrice is typically used along with a device (toothbrush) to clean the teeth and mouth. Mouthwashes are typically used to rinse the mouth after brushing by swishing and spitting the expectorate. These two products are used at a bathroom setting and are not convenient to use on the go. Consumers are increasingly looking for products that can provide oral care benefits but still are convenient to use on the go when needed, anywhere and at any time.

### OBJECTS AND SUMMARY OF THE INVENTION

The present disclosure aims at providing an oral care composition that is natural, portable, convenient to use on the go, cleans surfaces in the oral cavity well, minimizes harm to surfaces in the oral cavity, freshens breath, and generally provides comparable benefits to conventional liquid mouthwash, all while eliminating inconveniences associated with using a mouthwash or brushing teeth outside of a home bathroom setting.

The above objects of the present invention and others may be achieved by the present disclosure which in certain embodiments is directed to a mouthwash gummy, a method of preparing a mouthwash gummy, a method of cleaning a surface and/or treating a condition and/or freshening breath in an oral cavity of a subject with the mouthwash gummy, and/or a kit including the mouthwash gummy.

The mouthwash gummy disclosed herein may comprise a therapeutically effective amount of an active agent (e.g., an abrasive) for treatment of an oral condition and an excipient. The mouthwash gummy may be in a form of an elastic solid.

The mouthwash gummy disclosed herein may have be characterized in terms of its water activity, hardness, moisture content, pH, acceptance by consumers (e.g., consumer acceptance profile), or a combination thereof

Any of the mouthwash gummies disclosed herein may be suitable for cleansing a surface in a subject's oral cavity, refreshing a subject's breath, treating a condition in a subject's oral cavity, or any combination thereof. These goals may be attained in certain embodiments by retaining the mouthwash gummy in a subject's oral cavity for a certain duration ranging, e.g., from a few seconds to a few minutes.

In some embodiments, the present disclosure is directed to a kit comprising any of the mouthwash gummies disclosed herein enclosed in a suitable storage container (e.g., a bottle, blister pack, etc.).

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 depicts a plot illustrating consumer study results of a mouthwash gummy according to embodiments as compared to a mouthwash.

FIG. 2 depicts a plot illustrating water activity of a mouthwash gummy according to embodiments.

FIG. 3 depicts a plot illustrating hardness of a mouthwash gummy according to embodiments.

### DEFINITIONS

As used herein, the singular forms “a,” “an,” and “the” include plural references unless the context clearly indicates otherwise. Thus, for example, reference to “an abrasive” includes a single abrasive as well as a mixture of two or more different abrasives, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

As used herein, the terms “active agent” and “active ingredient” refer to any material that is intended to produce a therapeutic, prophylactic, or other intended effect, whether or not approved by a government agency for that purpose. These terms with respect to specific agents include all active agents, all pharmaceutically acceptable salts thereof, complexes, crystalline forms, co-crystals, ether, esters, hydrates, solvates, and mixtures thereof

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of an active agent. Exemplary medical conditions may include, without limitations, caries, gingivitis, hypersensitivity, plaque formation, calculus formation, tooth stains, halitosis, combinations thereof, and the like.

The terms “treatment of” and “treating” includes the lessening of the severity of or cessation of a condition or lessening the severity of or cessation of symptoms of a condition, e.g., an oral condition such as plaque.

The terms “prevention of” and “preventing” includes the avoidance of the onset of a condition, e.g., an oral condition such as plaque.

“Therapeutically effective amount” is intended to include an amount of an active agent, or an amount of the combination of active agents, e.g., to treat or prevent the condition, or to treat the symptoms of the condition, in a subject.

The phrase “pharmaceutically acceptable” refers to those compounds, materials, and/or compositions, within the scope of sound medical judgment, suitable for use in contact with the tissues of human beings and animals without excessive toxicity, irritation, allergic response, or other problems or complications commensurate with a reasonable benefit/risk ratio.

The term “conventional liquid mouthwash” refers to a solution that is designed and formulated to provide oral hygiene. Mouthwashes combine ingredients to target a variety of oral conditions such as plaque, gingivitis, bad breath and cavities.

### DETAILED DESCRIPTION

This disclosure is directed in certain embodiments to a natural product in a form of a gummy that can provide comparable cleansing and breath freshening benefits as a mouthwash while being portable enough to carry around and use on the go. The natural mouthwash gummy disclosed herein may be an edible composition for delivering into an individual's oral cavity one or more active agents that can cleanse the individual's oral cavity and/or freshen the individual's breath. A mouthwash product packaged into a gummy candy form provides consumers a number of advantages, including, portability, convenience, and improved oral health due to ease of use and a corresponding increased likelihood and/or frequency of use. Consumers may also be amenable to incorporating the mouthwash gummy disclosed herein due to their familiarity with gummies for other purposes (e.g., as a vitamin and/or nutritional supplement).

In certain embodiments, the instant disclosure may be directed to a mouthwash gummy comprising a therapeutically effective amount of an active agent for treatment of an oral condition and an excipient. The mouthwash gummy may be an elastic solid.

Suitable active agents for treating or prevent an oral condition may include, without limitations, one or more of, an abrasive, a probiotic, vitamins, a multivitamin, bioactives, mannitol, ammonium glycyrrhizate, caffeine, zinc gluconate, aesculus hippocastanum seed extract, hexamidine diisethionate, hydrolyzed linseed extract, pisum sativum extract, cyclodextrin, brassica campestris sterols, cetearyl alcohol, steroids, NSAIDs, a fluoride ion source (e.g., sodium F anti-cavity agent), polycarboxylate polymers, polyvinyl methyl ether/maleic anhydride (PVME/MA) copolymers, an arginine ester, a zinc ion source, a stannous ion source, delmopinol, tartar control agents, an antibacterial agent, triclosan and salts thereof, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, octapinol, nisin, a copper ion source, an essential oil, a furanone, anti-inflammatory agents, antiplaque agents, antioxidants, and a bacteriocins, honokiol, anti-attachment agents, proteinaceous agents, peptides, anesthetics, whitening agents, salts thereof, and combinations thereof

In certain embodiments, the active agent may comprise one or more of an abrasive, a probiotic, a multivitamin, mannitol, ammonium glycyrrhizate, caffeine, zinc gluconate, aesculus hippocastanum seed extract, hexamidine diisethionate, hydrolyzed linseed extract, pisum sativum extract, cyclodextrin, brassica campestris sterols, cetearyl alcohol, or any combination thereof

In one embodiment, the active agent may comprise an abrasive. The abrasive may be one or more of silica, clay, charcoal, magnesium aluminum phyllosilicate precipitated silicas, sodium metaphosphate, potassium metaphosphate, tricalcium phosphate, dihydrated dicalcium phosphate, aluminum silicate, calcined alumina, bentonite or other siliceous materials, particulate thermosetting resins, such as melamine, phenolic, and urea-formaldehydes, and cross-linked polyepoxides and polyesters, and combinations thereof

In one embodiment, the active agent may be an abrasive that is one or more of silica, clay, and/or charcoal.

In one embodiment, the active agent may comprise a combination of mannitol, ammonium glycyrrhizate, caffeine, zinc gluconate, and aesculus hippocastanum seed extract.

In one embodiment, the active agent may comprise hexamidine diisethionate.

In one embodiment, the active agent may comprise hydrolyzed linseed extract.

In one embodiment, the active agent may comprise a combination of pisum sativum (pea) extract and cyclodextrin.

In one embodiment, the active agent may comprise a bioactive. The bioactive may be, for instance, a combination of brassica campestris (rapeseed) sterols (e.g., about 50%) and cetearyl alcohol (e.g., about 50%).

One or more active agent(s) may be present, individually or in total (if more than one active agent is included), in the mouthwash gummy in a therapeutically effective amount ranging, e.g., from about 0.001 wt % to about 20 wt %, from about 0.01 wt % to about 10 wt %, from about 0.1 wt % to about 5 wt %, from about 0.2 wt % to about 3 wt %, or from about 0.3 wt % to about 2 wt %, based on total weight of the mouthwash gummy. The mouthwash gummy may have a total weight ranging from about 0.5 g to about 10 g, from about 1 g to about 8 g, from about 2 g to about 5 g, or about 3 g to about 4 g. The therapeutically effective amount of active agent in the mouthwash gummy may be within about 30%, within about 20%, within about 10%, within about 5%, within about 2%, within about 1%, within about 0.5%, or equivalent (within 0%) of the amount of active agent in a single dose of a conventional liquid mouthwash. The term “within,” as used herein, refers to Δ as calculated below. The percent difference (Δ) is the amount of active agent in the mouthwash gummy (A_{gummy}) as compared to the amount of active agent in a single dose of a conventional liquid mouthwash (A_{liquid}) may be calculated as follows:

$$Δ = \frac{\vert {A}_{g u m m y} - {A}_{liquid} \vert}{{A}_{liquid}} \cdot 100 \%$$

A single dose of a conventional liquid mouthwash may have a volume of about 5 ml to about 50 ml, about 10 ml to about 40 ml, or about 15 ml to about 30 ml.

In some embodiments, suitable excipients to be incorporated in the mouthwash gummy disclosed herein may include, without limitations, a flavoring agent, a sweetener, a colorant, a dye, a pigment, an antioxidant, a binder, a solvent, a humectant, a viscosity modifier, a foaming agent, a solubilizer, a desensitizing agent, a bleaching agent, an anti-cavity agent, a stain prevention agent, a complexing agent, a preservative, a pH adjusting agent, an opacifier, a breath freshening agent, a soothing agent, a surfactant, a propellant, a coupling agent, a chelating agent, an abrasive, an additional active agent, a gelling agent, an effervescence material, and combinations thereof. In certain embodiments, the excipients that are included in the mouthwash gummy disclosed herein are natural so as to minimize irritation and/or allergic reactions to the consumer.

The mouthwash gummy may include a flavoring agent or a mixture of flavoring agents including natural or synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils, essences, limonene, menthone, carvone, menthol, anethole, eucalyptol, anethole, eugenol, cassia, oxanone, a-irisone, propenyl guaiethol, thymol, linalool, benzaldehyde, cinnamaldehyde, N-ethyl-p-menthan-3-carboxamine, N,2,3 -trimethyl-2-isopropylbutanamide, 3-1-menthoxypropane-1,2-diol, cinnamaldehyde glycerol acetal (CGA), methone glycerol acetal (MGA), cineole, and combinations thereof

Flavoring agent(s) may be present, individually or in total (if more than one flavoring agent is included), in the mouthwash gummy in an amount ranging from about 0.1 wt % to about 5 wt %, from about 0.2 wt % to about 1.5 wt %, or from about 0.5 wt % to about 1 wt % (calculated as the total weight of flavoring agent(s) in the mouthwash gummy divided by the total weight of the mouthwash gummy).

Colorants and/or dyes and/or pigments may be added to the mouthwash gummy in some embodiments. Suitable colorants and/or dyes and/or pigments may include, but not be limited to, colors such as e.g., white, black, yellow, blue, green, pink, red, orange, violet, indigo, brown, and combinations thereof, pigments such as, e.g., Timica Extra Large Sparkles, titanium dioxide and chromium oxide greens, ultramarine blues and pinks and ferric oxides.

Colorants and/or dyes and/or pigments may be present, individually or in total (if more than one colorant and/or dye and/or pigment is included), in the mouthwash gummy in an amount ranging from about 0.01 wt % to about 1 wt %, from about 0.01 wt % to about 0.5 wt %, from about 0.02 wt % to about 0.4 wt %, or from about 0.03 wt % to about 0.3 wt % (calculated as the total weight of colorants and/or dyes and/or pigments in the mouthwash gummy divided by the total weight of the mouthwash gummy).

Antioxidants may be added to the mouthwash gummy in some embodiments. Suitable antioxidants may include, but not be limited to, natural antioxidants such as tocopherol and tocopherol acetate. In one embodiment, the antioxidant in the mouthwash gummy may comprise vitamin E.

Antioxidants may be present, individually or in total (if more than one antioxidant is included), in the mouthwash gummy in an amount ranging from about 0.1 wt % to about 5 wt %, from about 0.3 wt % to about 3 wt %, or from about 0.5 wt % to about 2 wt % (calculated as the total weight of antioxidants in the mouthwash gummy divided by the total weight of the mouthwash gummy).

The mouthwash gummy of the invention may contain a binder that could contribute to the rheology of the mouthwash gummy and to the feel of the mouthwash gummy in the oral cavity. Suitable binders include, without limitations, polyvinylpyrrolidone (PVP), marine colloids, carboxyvinyl polymers, starches, cellulosic polymers such as hydroxyethylcellulose, carboxymethylcellulose (carmellose), hydroxypropyl methyl cellulose, hydroxyethylpropylcellulose, hydroxybutyl methyl cellulose, and salts thereof (e.g., carmellose sodium), natural gums such as karaya, xanthan, carrageenans, gellan gum, locust bean gum, gum arabic and tragacanth, chitosan, colloidal magnesium aluminum silicate, and colloidal silica.

Binders may be present, individually or in total (if more than one binder is included), in the mouthwash gummy in an amount ranging from about 0.01 wt % to about 3 wt %, from about 0.01 wt % to about 1 wt %, or from about 0.01 wt % to about 0.2 wt % (calculated as the total weight of binders in the mouthwash gummy divided by the total weight of the mouthwash gummy.

Solvents that may be included in the mouthwash gummy disclosed herein may include, without limitations, water, polyhydric alcohols (e.g., glycerin), 1,3-butylene glycol, propylene glycol, hexylene glycol, propane diol, ethylene glycol, diethylene glycol, dipropylene glycol, diglycerin, sorbitol, other sugars which are liquid at room temperature, water soluble alkoxylated nonionic polymers such as polyethylene glycol, and combinations thereof. In one embodiment, the solvent in the mouthwash gummy may comprise one or more of water, sorbitol, and combinations thereof

Solvents may be present, individually or in total (if more than one solvent is included), in the mouthwash gummy in an amount ranging from about 5 wt % to about 95 wt %, from about 25 wt % to about 85 wt %, from about 35 wt % to about 75 wt %, from about 5 wt % to about 15 wt %, or from about 20 wt % to about 60 wt % (calculated as the total weight of solvents in the mouthwash gummy divided by the total weight of the mouthwash gummy).

Humectant(s), such as low molecular weight polyethylene glycol (e.g., PEG6-PEG12), may be present, individually or in total (if more than one humectant is included), in the mouthwash gummy in an amount of up to about 7 wt %, up to about 5 wt %, up to about 3 wt %, up to about 1 wt %, or up to about 0.1 wt % (calculated as the total weight of humectants in the mouthwash gummy divided by the total weight of the mouthwash gummy).

The mouthwash gummy may include a sweetener capable of providing a palatable and pleasurable factor to the user, and/or capable of masking undesirable flavors present in the dosage form. The sweetener may be the kind that does not enhance the severity of the oral condition (e.g., a sweetener than does not cause and/or enhance cavities). In some embodiments, the sweetener may also provide a therapeutic effect for treatment of the oral condition.

Exemplary sweeteners that may be in the mouthwash gummy may include, but not be limited to, one or more artificial sweeteners, one or more natural sweeteners, one or more soluble fibers (e.g., for a sugar free for mouthwash gummy formulation), or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like. Examples of soluble fibers that can be incorporated into the mouthwash gummy, alone or in combination with other sweeteners, may include, but not be limited to, soluble fibers from tapioca, fruit (e.g., pears, nectarines, apples, and the like), starch, nuts (such as oats, hazelnuts, barley and the like), and legume sources.

In one embodiment, the sweetener in the mouthwash gummy may comprise xylitol.

Sweeteners may be present, individually or in total (if more than one sweetener is included), in the mouthwash gummy in an amount ranging from about 1 wt % to about 20 wt %, from about 3 wt % to about 18 wt %, from about 5 wt % to about 15 wt %, from about 7 wt % to about 13 wt %, or from about 9 wt % to about 11 wt % (calculated as the total weight of sweeteners in the mouthwash gummy divided by the total weight of the mouthwash gummy).

Surfactants may be incorporated in the mouthwash gummy disclosed herein. The surfactants that can be included in the mouthwash gummy may be anionic, nonionic, or amphoteric compounds.

Suitable examples of anionic surfactants are one or more of higher alkyl sulfates such as potassium or sodium lauryl sulfate, higher fatty acid monoglyceride monosulfates, such as the salt of the monosulfated monoglyceride of hydrogenated coconut oil fatty acids, alkyl sulfonates such as sodium dodecyl benzene sulfonate, higher fatty sulfoacetates, higher fatty acid esters of 1,2 dihydroxypropane sulfonate.

Examples of water soluble nonionic surfactants are condensation products of ethylene oxide with various hydrogen-containing compounds that are reactive therewith and have long hydrophobic chains (e.g., aliphatic chains of about 12 of 20 carbon atoms), which condensation products contain hydrophilic polyoxyethylene moieties, such as condensation products of poly (ethylene oxide) with fatty acids, fatty alcohols, fatty amides and other fatty moieties, and with propylene oxide and polypropylene oxides, e.g., Pluronic materials such as Pluronic F127.

Exemplary suitable alkyl polyglycoside (APG) surfactant(s) that may be used in the mouthwash gummy disclosed herein may comprise APG C8-C10, APG C10-C16, decyl glucoside, coco-glucoside, anionic APG carboxylate, sodium lauryl glucose carboxylate, lauryl glucoside, D-glucopyranose (oligomeric, C10-16 glycosides, carboxymethyl ethers, sodium salts), C12-C16 fatty alcohol glycoside, and combinations thereof. Exemplary APG surfactant(s) that may be used may have an industry designation of Plantaren® 2000 N UP/MB, Plantapon® LGC Sorb, Plantaren® 1200 N UP/MB, and Plantaren® 818 UP/MB.

In one embodiment, the surfactant in the mouthwash gummy may be a combination of sodium lauryl glucose carboxylate and lauryl glucoside.

Surfactants may be present, individually or in total (if more than one surfactant is included) in the mouthwash gummy in an amount ranging from about 0.01 wt % to about 5 wt %, from about 0.01 wt % to about 2 wt %, or from about 0.01 wt % to about 0.5 wt % (calculated as the total weight of surfactants in the mouthwash gummy divided by the total weight of the mouthwash gummy).

Exemplary gelling agent(s) that may be issued in the mouthwash gummy disclosed herein may comprise pectins, starches, and gelatin forms derived from animals or from plants (e.g., pork gelatin) or a different gelling agent that would be suitable for consumption by individuals who do not consume meat products (e.g., vegeterians). The pectin in the mouthwash gummy disclosed herein may include, e.g., high methoxyl pectin, low methoxyl pectin, or a combination thereof. In some embodiments, the pectin is amidated pectin. In other embodiments, the pectin is non-amidated pectin. In certain embodiments, the pectin is a combination of amidated pectin and non-amidated pectin. The gelatin in the mouthwash gummy disclosed herein may include Type A gelatin, Type B gelatin, a hide or skin gelatin (e.g., calf skin, pig skin) and/or a bone gelatin (e.g., calf bone, pig bone) used alone or in combination.

Gelling agent(s) may be present, individually or in total (if more than one gelling agent is included) in the mouthwash gummy in an amount ranging from about 1 wt % to about 20 wt %, from about 3 wt % to about 15 wt %, or from about 5 wt % to about 10 wt % (calculated as the total weight of gelling agents in the mouthwash gummy divided by the total weight of the mouthwash gummy).

The total amount of all excipients in the mouthwash may range from about 80 wt % to about 99.999 wt %, from about 90 wt % to about 99.99 wt %, from about 95 wt % to about 99.9 wt %, from about 97 wt % to about 99.8 wt %, from about 98 wt % to about 99.7 wt % (calculated as the total weight of all excipients in the mouthwash gummy divided by the total weight of the mouthwash gummy).

In some embodiments, effervescent materials may be added to the mouthwash gummy to provide fizz. Suitable effervescent materials may include compounds which evolve gas, e.g., by means of a chemical reaction which takes place upon exposure of the effervescent material to water and other fluids (for instance, produce bubbles upon mixing with saliva). The acid sources may be any which are safe for human consumption and may generally include food acids, acid and hydrite antacids such as, for example, citric, tartaric, amalic, fumeric, adipic, and succinics. Carbonate sources include dry solid carbonate and bicarbonate salt such as, sodium bicarbonate, sodium carbonate, potassium bicarbonate and potassium carbonate, magnesium carbonate and the like. In one embodiment, the effervescent material is sodium bicarbonate.

Effervescent materials may be present, individually or in total (if more than one effervescent material is included), in the mouthwash gummy in an amount ranging from about 0.01 wt % to about 5 wt %, from about 0.1 wt % to about 3 wt %, or from about 0.2 wt % to about 2 wt % (calculated as the total weight of effervescent materials in the mouthwash gummy divided by the total weight of the mouthwash gummy).

The pH of the mouthwash gummy disclosed herein may range from about 1 to about 5, from about 2 to about 4, or from about 2.5 to about 3.5. The pH of the mouthwash gummy may be measured by dissolving one part of the mouthwash gummy in three parts of water (1:3 (weight gummy):(weight water)) and using a pH meter equipped with a glass combined electrode. The pH of the mouthwash gummy may be adjusted with a pH adjusting material such as, without limitations, citric acid, hydrochloric acid, sodium hydroxide, etc.

The pH adjusting agent may be present, individually or in total (if more than one pH adjusting agent is included), in the mouthwash gummy in an amount ranging from about 0.01 wt % to about 2 wt %, from about 0.1 wt % to about 1 wt %, or from about 0.2 wt % to about 0.5 wt % (calculated as the total weight of pH adjusting agent(s) in the mouthwash gummy divided by the total weight of the mouthwash gummy).

The hardness of the mouthwash gummy disclosed herein may range from about 0.3N to about 0.9N, from about 0.5N to about 0.95N, or from about 0.7N to about 0.9N measured at room temperature by compressing the mouthwash gummy with a probe until the mouthwash gummy is deformed. A mouthwash gummy with a hardness value that is below about 1.0N may be indicative of easiness to chew.

The water activity of the mouthwash gummy disclosed herein may range from about 0.2 to about 0.9, from about 0.4 to about 0.8, or from about 0.3 to about 0.7. The water activity may be measured by loading 3 gram of sample into a benchtop water activity meter. The dew point sensor in the instrument can accurately predict water activity. The water activity measured the unbound water in the mouthwash gummy. Less unbound water may be indicative of good long term stability.

The moisture content of the mouthwash gummy disclosed herein may range from about 15 to about 30, from about 18 to about 27, from about 20 to about 25. The moisture content may be measured at room temperature using Karl Fischer titration. The moisture content in the mouthwash gummy may be indicative of how chewy the mouthwash gummy will become. The higher the moisture content, the less chewy the mouthwash gummy is. The moisture content in the mouthwash gummy may be adjusted to as to attain a target chewability.

The mouthwash gummy disclosed herein may dissolve in a subject's saliva in about 20 seconds to about five minutes, about 25 second to about two minutes, or from about 30 seconds to about one minute.

The mouthwash gummy may be designed to perform in a similar manner as a conventional mouthwash formulation by providing one or more of a similar flavor, a similar breath freshening effect, a similar cleansing effect, or other similar effects. The effects of a conventional liquid mouthwash and of the mouthwash gummy disclosed herein may be evaluated through a first consumer acceptance profile and a second consumer acceptance profile, respectively. The first consumer acceptance profile and the second consumer acceptance profile may be similar or substantially similar.

A consumer acceptance profile may be prepared by questioning a preselected number of consumers. The consumers may be asked to evaluate their perception of one or more parameters related to a tested product on a scale of 1-10. The one or more parameters related to evaluation of a mouthwash gummy and/or a conventional liquid mouthwash may vary and may include, without limitations, flavor acceptance, sweet flavor, sour flavor, salty flavor, bitter flavor, soapy flavor, spicy flavor, peppermint flavor, cooling sensation, refreshing sensation, astringent sensation, cleanliness, and any combination thereof

The similarity between two consumer acceptance profiles may be evaluated based on the difference in value for one or more of the tested parameters. For instance, the first consumer acceptance profile (performed for a conventional liquid mouthwash) may be within about 30%, within about 25%, within about 20%, within about 15%, within about 10%, or within about 5% from the second consumer acceptance profile (performed for a mouthwash gummy) in one or more of the tested parameters (such as flavor acceptance, sweet flavor, sour flavor, salty flavor, bitter flavor, soapy flavor, spicy flavor, peppermint flavor, cooling sensation, refreshing sensation, astringent sensation, cleanliness, and any combination thereof). The term “within,” as used herein may be designated as Δ_{acceptance} and calculated as explained below.

The percent difference (Δ_{acceptance}) in the consumer acceptance of a parameter with respect to the mouthwash gummy (P_{gummy}) as compared to the consumer acceptance of the same parameter with respect to the conventional liquid mouthwash (P_{liquid}) may be calculated as follows:

$${Δ}_{a c c e p t a n c e} = \frac{\vert {P}_{g u m m y} - {P}_{liquid} \vert}{{P}_{l i quid}} \cdot 100 \%$$

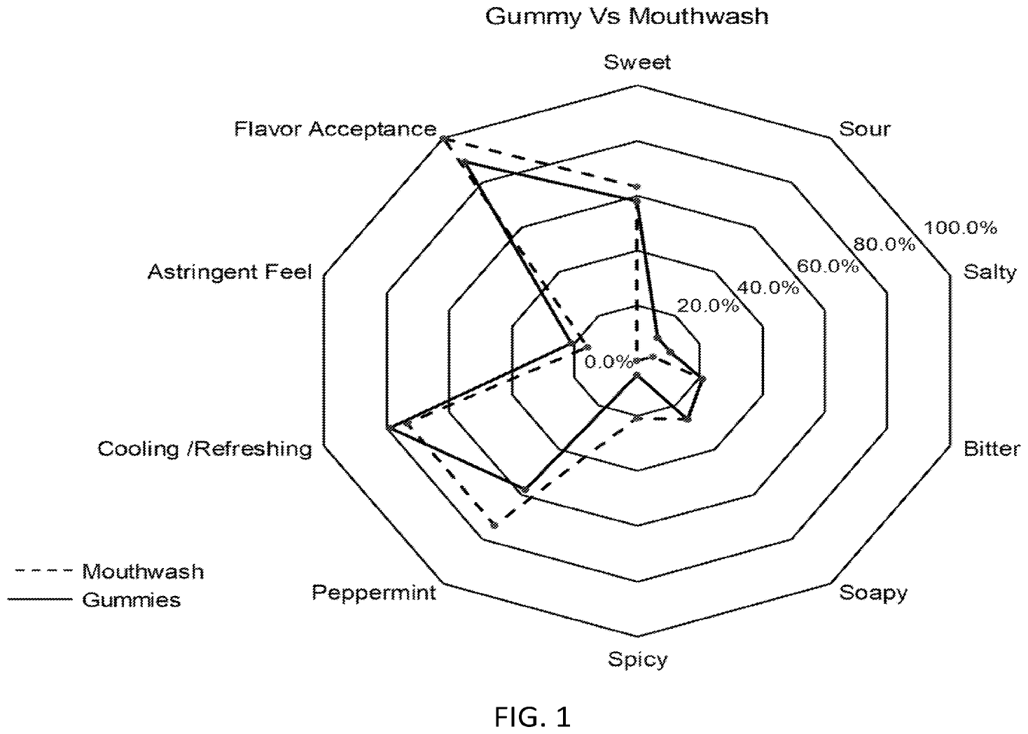

A consumer acceptance profile may also be represented in a graphical representation as illustrated in FIG. 1. In certain embodiments, the similarity between two or more consumer acceptance profiles may be determined based on the similarity in the graphical shape of each of the evaluated consumer acceptance profiles. In one embodiment, the first consumer acceptance profile (performed for a conventional liquid mouthwash) may have a first graphical shape that is substantially similar to a second graphical shape of a second consumer acceptance profile (performed for a mouthwash gummy). For instance, in FIG. 1 the graphical shape of both the first consumer acceptance study and of the second consumer acceptance study resembles a similarly tilted “Y” shape.

The oral conditions that may be treated with the mouthwash gummy described herein include, without limitations, caries, gingivitis, hypersensitivity, plaque formation, calculus formation, tooth stains, halitosis, or any combinations thereof. A conventional liquid mouthwash may be one or more of antiseptic, acid neutralizer, moisturizer for the oral cavity, provide anti-plaque benefits, and reduce bad breath. Similar benefits may be experienced with the mouthwash gummy described herein.

Some embodiments of the instant disclosure may be directed to a method for preparing the oral mouthwash gummy disclosed herein. The method may comprise combining a therapeutically effective amount of an active agent suitable for treatment of an oral condition and an excipient(s) to form an elastic solid.

Some embodiments of the instant disclosure may be directed to a method of cleaning a surface and/or treating an oral condition in an oral cavity of a subject and/or refreshing a subject's breath. These methods may comprise retaining the mouthwash gummy described herein in an oral cavity for a duration of time. Ultimately, the surface in the oral cavity of the subject may be cleaned and/or the subject's oral condition may be treated and/or the subject's breath may be refreshed.

The surface in the oral cavity of a subject may encompass, for instance, teeth, gums, tongue, cheeks, and combinations thereof

The duration for retaining the mouthwash gummy in a subject's oral cavity may range from about 10 seconds to about 10 minutes, from about 15 seconds to about 8 minutes, from about 20 seconds to about 5 minutes, from about 25 seconds to about 2 minutes, or from about 30 seconds to about 1 minutes.

Retaining the mouthwash gummy in an oral cavity may encompass chewing the mouthwash gummy, dissolving the mouthwash gummy in the oral cavity, holding the mouthwash gummy in the oral cavity, or any combination thereof

Some embodiments of the instant disclosure may be directed to a kit comprising the mouthwash gummy disclosed herein enclosed in a suitable storage container. A suitable container may be a bottle, a blister pack, a bag, a sleeve wrap, and combinations thereof. The mouthwash gummy may be packaged in bulk or individually. The storage containers may vary in sizes and may be suitable for enclosing a varied number of mouthwash gummies.

### ILLUSTRATIVE EXAMPLES

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1: Mouthwash Gummy Compositions

In this example, a mouthwash gummy was prepared as summarized in Table 1 below. The mouthwash gummy was prepared by blending the ingredients together at high temperature. The texture and level of chewiness in the gummy was adjusted by the amount of gelatin used in the gummy. Once the ingredients are blended. The hot blend liquid is then transferred onto a starch mogul molding and allowed to cool off and solidify overnight. Once the mixture is the set the gummies are removed from the moldings. Each gummy has a weight of about 3 to about 4 grams with active delivery that is equivalent to about 15 ml of mouthwash. As reference, a non-limiting example of a conventional liquid mouthwash formulation is disclosed in Table 2.

TABLE 1
Mouthwash Gummy Composition

| | | Concentration |
|---|---|---|
| Ingredients | Function | (wt % ranges) |
| 250 Bloom Gelatin | Gelling Agent | 5.0-10.0 |
| Water | Solvent | 5.0-15.0 |
| Natural Sorbitol | Solvent | 20-60 |
| Xylitol | Sweetener | 5.0-15.0 |
| Vitamin E | Antioxidant | 0.5-2 |
| Natural Color 1% solution | Colorant | 0.01-1 |
| Natural Flavor | Flavoring Agent | 0.2-1.5 |
| Xanthan Gum | Binder | 0.01-0.2 |
| Plantapon LGC Sorb | Surfactant | 0.01-0.5 |
| Bioactive (Phytosoothe LS 9766) | Active Agent | 0.1-2 |
| Citric Acid | pH adjusting agent | 0.01-0.5 |
| Silica/Clay/Charcoal | Abrasive | 0.2-3 |

TABLE 2
Exemplary Liquid Mouthwash Formulation

| Ingredients | Concentration (wt %) |
|---|---|
| Water | 84.13 |
| Poloxamer 407 | 0.6 |
| Sodium Saccharin | 0.05 |
| Sodium Fluoride | 0.05 |
| Sodium Benzoate | 0.5 |
| Sorbitol | 3 |
| Polyvinylpyrrolidone (PVP) | 1.5 |
| FD&C Blue No. 1 (1% solution) | 0.05 |
| PEG-40 Hydrogenated Castor Oil | 0.05 |
| Citric Acid | 0.02 |
| Flavor | 0.5 |
| 1,2 Propylene Glycol Care | 7.8 |

### Example 2: Gummy Water Activity

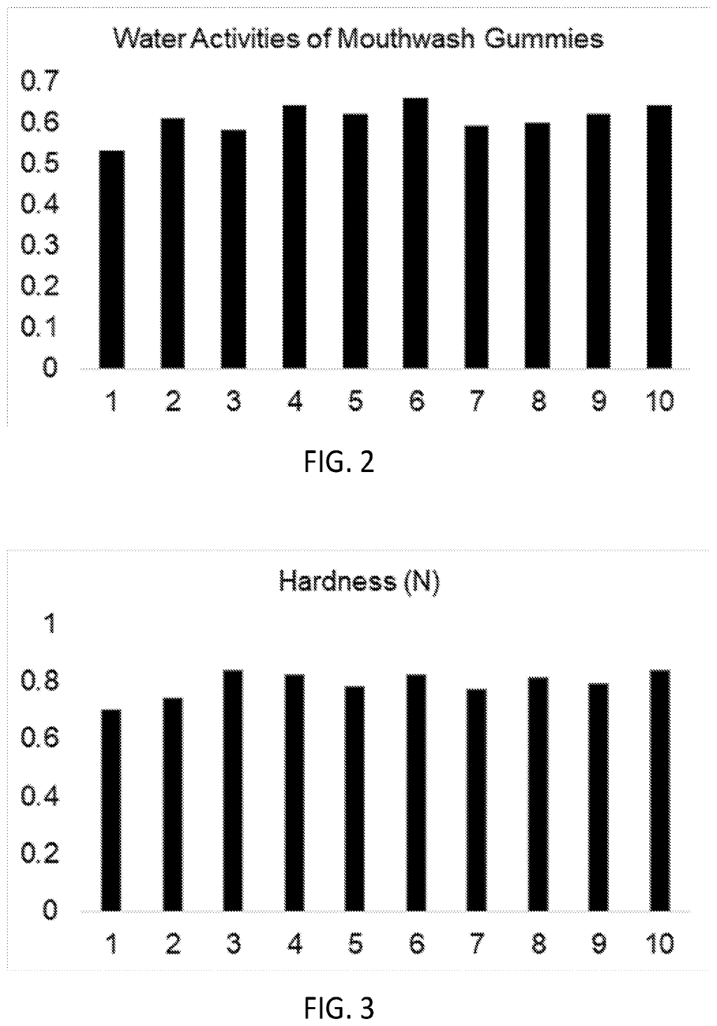

Unbound water in the mouthwash gummy disclosed herein was measured and is summarized in Table 3 below and in corresponding FIG. 2. The average unbound water in the tested gummies was about 0.609. Less unbound water is an indication of long-term stability. Stability is measured by aging the gummies and evaluating the micro-organism growth of an aged gummy. Higher water activity is believed to increase chances of bacterial growth and thus lead to an unstable product.

TABLE 3
Gummy Water Activity

| Sample # | Water activity |
|---|---|
| 1 | 0.53 |
| 2 | 0.61 |
| 3 | 0.58 |
| 4 | 0.64 |
| 5 | 0.62 |
| 6 | 0.66 |
| 7 | 0.59 |
| 8 | 0.6 |
| 9 | 0.62 |
| 10 | 0.64 |
| Ave | 0.609 |

### Example 3: Gummy Hardness

Gummy hardness (N) was measured to gauge the easiness to chew the mouthwash gummy. The average hardness of the tested gummies was 0.80N. A number below 1.0N is an indication of easiness to chew. The hardness was tested at room temperature (about 23° C. to about 24° C.) by compressing the gummy samples with a probe until they were deformed. The measured hardness for each tested gummy sample are summarized in Table 4 below and in corresponding FIG. 3. The gummy hardness and water activity depend on, among other factors, the drying time, the level of water in the formulation, and the gelatin levels in the formulation.

TABLE 4
Gummy Hardness

| Sample # | Hardness (N) |
|---|---|
| 1 | 0.7 |
| 2 | 0.74 |
| 3 | 0.84 |
| 4 | 0.82 |
| 5 | 0.78 |
| 6 | 0.82 |
| 7 | 0.77 |
| 8 | 0.81 |
| 9 | 0.79 |
| 10 | 0.84 |
| Ave | 0.791 |

### Example 4: Moisture

Moisture was determined by Karl Fischer titration. All measurements were conducted in room temperature (about 24° C.). The moisture content measures the level of liquidness after drying of the gummy is complete. The moisture content may depend on water and other humectants. Moisture content is indicative of how chewy the gummy will become. The higher the moisture content the less chewy the gummy is. The measured moisture content over 28 hours are summarized in Table 5 below.

TABLE 5
Moisture Content

| | Moisture |
|---|---|
| Hours | (g/100 g) |
| 12 | 22 |
| 16 | 22 |
| 20 | 23 |
| 24 | 23 |
| 28 | 22 |
| Ave | 22.4 |

### Example 4: pH

The pH was measured using a pH meter equipped with a glass combined electrode. The samples were sliced, mixed with water at 25° C. (1:3, w:w), and stirred until completely dissolved prior to measuring the pH. The measured pH over 28 hours is summarized in Table 6 below.

TABLE 6
Moisture Content

| Hours | pH |
|---|---|
| 12 | 3.3 |
| 16 | 2.8 |
| 20 | 3 |
| 24 | 2.9 |
| 28 | 3.2 |
| Ave | 3.04 |

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A mouthwash gummy comprising
a therapeutically effective amount of an active agent for treatment of an oral condition; and
an excipient,
wherein the mouthwash gummy is an elastic solid.

2. The mouthwash gummy of claim 1, having a water activity of about 0.2 to about 0.9.

3. The mouthwash gummy of claim 1, having a hardness of about 0.3N to about 0.9N, measured at room temperature by compressing the mouthwash gummy with a probe until the mouthwash gummy is deformed.

4. The mouthwash gummy of claim 1, having a moisture content of about 15 to about 30, measured at room temperature using Karl Fischer titration.

5. The mouthwash gummy of claim 1, having a pH of about 1 to about 5, measured by dissolving one part of the mouthwash gummy in three parts of water and using a pH meter equipped with a glass combined electrode.

6. The mouthwash gummy of claim 1, wherein the mouthwash gummy exhibits a first consumer acceptance profile and a liquid mouthwash exhibits a second consumer acceptance profile, and wherein the first consumer acceptance profile is substantially similar to the second consumer acceptance profile.

7. The mouthwash gummy of claim 1, wherein the mouthwash gummy dissolves in a subject's saliva in about 20 seconds to about five minutes.

8. The mouthwash gummy of claim 1, wherein the active agent comprises an abrasive, a probiotic, a multivitamin, mannitol, ammonium glycyrrhizate, caffeine, zinc gluconate, aesculus hippocastanum seed extract, hexamidine diisethionate, hydrolyzed linseed extract, pisum sativum extract, cyclodextrin, brassica campestris sterols, cetearyl alcohol, or any combination thereof

9. (canceled)

10. The mouthwash gummy of claim 1, wherein the therapeutically effective amount of the active agent is about 0.001 wt % to about 20 wt %, based on total weight of the mouthwash gummy.

11. The mouthwash gummy of claim 1, wherein the oral condition comprises caries, gingivitis, hypersensitivity, plaque formation, calculus formation, tooth stains, halitosis, or any combinations thereof.

12. The mouthwash gummy of claim 1, wherein the first consumer acceptance profile has a first graphical shape that is substantially similar to a second graphical shape of the second consumer acceptance profile.

13. The mouthwash gummy of claim 1, wherein the first consumer acceptance profile and the second consumer acceptance profile are based on flavor acceptance, sweet flavor, sour flavor, salty flavor, bitter flavor, soapy flavor, spicy flavor, peppermint flavor, cooling sensation, refreshing sensation, astringent sensation, cleanliness, or any combination thereof.

14. (canceled)

15. The mouthwash gummy of claim 1, further comprising an excipient comprising one or more of a flavoring agent, a sweetener, a colorant, a dye, a pigment, an antioxidant, a binder, a solvent, a humectant, a viscosity modifier, a foaming agent, a solubilizer, a desensitizing agent, a bleaching agent, an anti-cavity agent, a stain prevention agent, a complexing agent, a preservative, a pH adjusting agent, an opacifier, a breath freshening agent, a soothing agent, a surfactant, a propellant, a coupling agent, a chelating agent, an abrasive, an additional active agent, an effervescence material, or combinations thereof

16. (canceled)

17. The mouthwash gummy of claim 15, wherein the sweetener is present in the mouthwash gummy at about 5 wt % to about 15 wt %, based on total weight of the mouthwash gummy.

18. (canceled)

19. The mouthwash gummy of claim 15, wherein the solvent is present in the mouthwash gummy at a concentration of from about 75 wt % to about 95 wt % based on total weight of the mouthwash gummy.

20. A method for cleaning a surface in an oral cavity of a subject comprising retaining the mouthwash gummy of claim 1 in the oral cavity of the subject, wherein the surface is cleaned.

21. A method for treating a condition in an oral cavity of a subject comprising retaining the mouthwash gummy of claim 1 in the oral cavity of the subject, wherein the condition is treated.

22. A method for refreshing a subject's breath comprising retaining the mouthwash gummy of claim 1 in the oral cavity of the subject, wherein the subject's breath is refreshed.

23. (canceled)

24. (canceled)

25. A method for preparing a mouthwash gummy according to claim 1, comprising combining the active agent for treatment of the oral condition and the excipient.

26. A kit comprising a storage container and a mouthwash gummy according to claim 1 enclosed in the storage container.
